(19) United States
(12) Plant Patent Application Publication
(10) Pub. No.: US 2020/0305326 P1
(43) Pub. Date: Sep. 24, 2020

(54) Chrysanthemum plant named 'DLFSALM2'

(50) ;

(71) Applicant: Arie Gerard Post, Delft (NL)

(72) Inventor: Arie Gerard Post, Delft (NL)

(73) Assignee: Deliflor Royal Ties B.V., Maasdijk (NL)

(21) Appl. No.: 16/873,324

(22) Filed: Mar. 19, 2020

Related U.S. Application Data
(60) Provisional application No. 62/919,611, filed on Mar. 20, 2019.

(51) Int. Cl. A01H 6/14

(52) U.S. Cl. PLT/289

(57) ABSTRACT

A new and distinct cultivar of Chrysanthemum plant named ‘DLFSALM2’, characterized by its upright plant habit; vigorous growth habit; dark green-colored leaves; uniform flowering habit; and excellent postproduction longevity.

Botanical designation: Chrysanthemum X morifolium.

Cultivar denomination: ‘DLFSALM2’.

### BACKGROUND OF THE INVENTION

The present invention relates to a new and distinct cultivar of Chrysanthemum plant, botanically known as Chrysanthemum x morifolium, typically grown as a cut flower Chrysanthemum and hereinafter referred to by the name ‘DLFSALM2’.

The new Chrysanthemum plant is a product of a planned breeding program conducted by the Inventor in Maasdijk, The Netherlands. The objective of the breeding program is to create new cut flower Chrysanthemum plants with numerous attractive inflorescences.

The new Chrysanthemum plant is a naturally-occurring whole plant mutation of Chrysanthemum x morifolium ‘Delisalma’, not patented. The new Chrysanthemum plant was discovered and selected as a single flowering plant from within a population of plants of ‘Delisalma’ in a controlled greenhouse environment in Maasdijk, The Netherlands in June, 2012.

Asexual reproduction of the new Chrysanthemum plant by vegetative terminal cuttings since June, 2012 has shown that the unique features of this new Chrysanthemum plant are stable and reproduced true to type in successive generations of asexual reproduction.

### SUMMARY OF THE INVENTION

Plants of the new Chrysanthemum have not been observed under all possible combinations of environmental conditions and cultural practices. The phenotype may vary somewhat with variations in environmental conditions such as temperature, daylength and light intensity, without, however, any variance in genotype.

The following traits have been repeatedly observed and are determined to be the unique characteristics of ‘DLFSALM2’. These characteristics in combination distinguish ‘DLFSALM2’ as a new and distinct Chrysanthemum plant:

1. Upright plant habit; vigorous growth habit.

2. Dark green-colored leaves.

3. Uniform flowering habit.

4. Strong upright flowering stems typically grown as a single-stem (disbudded) cut flower.

5. Large decorative-type inflorescences with bright yellow-colored ray florets.

6. Relatively resistant to Fusarium oxysporum f.sp. chrysanthemi and Verticillium dahliae.

### BRIEF DESCRIPTION OF THE PHOTOGRAPHS

The accompanying photographs illustrate the overall appearance of the new Chrysanthemum plant showing the colors as true as it is reasonably possible to obtain in colored reproductions of this type.

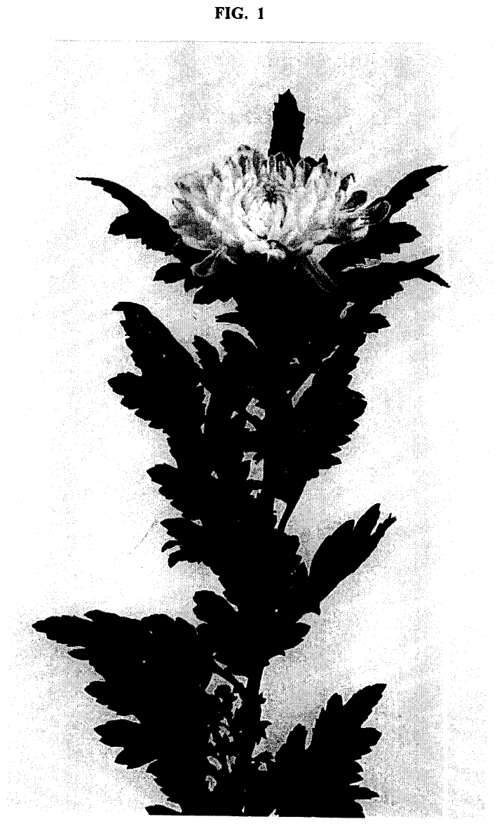

The photograph on the first sheet (FIG. 1 of 2) comprises a side perspective view of a typical flowering stem of ‘DLFSALM2’ grown as a disbud-type cut flower.

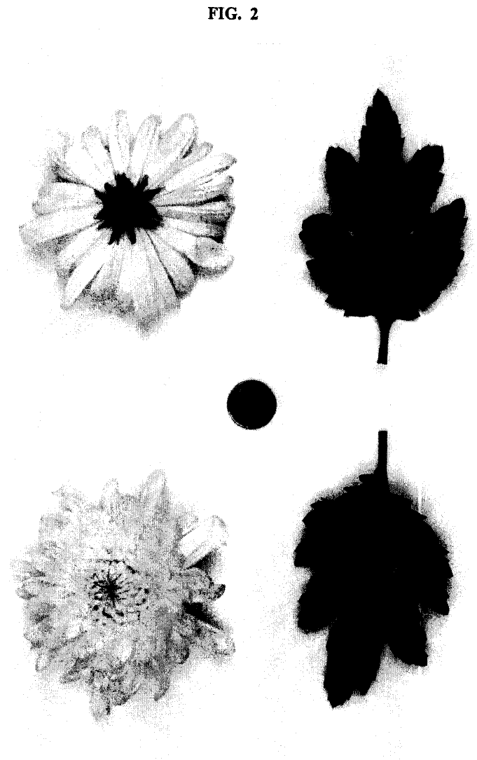

The photograph on the second sheen (FIG. 2 of 2) is a close-up view of lower (top of the photographic sheet) and upper (bottom of the photographic sheet) surfaces of typical inflorescences (left) and leaves (right).

### DETAILED BOTANICAL DESCRIPTION

The aforementioned photographs and following observations and measurements describe plants grown during the spring in ground beds in a glass-covered greenhouse in Maasdijk, The Netherlands and under cultural practices typical of commercial cut Chrysanthemum production. Plants were initially given long day/short night treatments followed by short day/long night treatments to induce flower initiation and development. During the production of the plants, day temperatures ranged from 18° C. to 22° C., night temperatures ranged from 20° C. to 25° C. and light levels averaged 8 klux. Plants were grown as single-stem disbud-type plants and were ten weeks old when the photographs and the description were taken. In the following description, color references are made to The Royal Horticultural Society Colour Chart, 2015 Edition, except where general terms of ordinary dictionary significance are used.
Botanical classification: Chrysanthemum X morifolium ‘DLFSALM2’.
Parentage: Naturally-occurring whole plant mutation of Chrysanthemum x morifolium ‘Delisalma’, not patented.
Propagation:
   Type.—Terminal vegetative cuttings.
   Time to initiate roots, summer.—About six days at temperatures about 24° C.
   Time to initiate roots, winter.—About eight days at temperatures about 22° C.
   Time to produce a rooted young plant, summer.—About twelve days at temperatures about 24° C.
   Time to produce a rooted young plant, winter.—About two weeks at temperatures about 22° C.
   Root description.—Fine, fibrous; typically creamy white in color, actual color of the roots is dependent on substrate composition, water quality, fertilizers, substrate temperature and physiological age of roots.
   Rooting habit.—Freely branching, medium density.
Plant description:
   Plant and growth habit.—Herbaceous decorative-type cut flower that is typically grown as a single stem disbud-type; upright plant habit; vigorous growth habit and moderate to rapid growth rate.
Pathogen & pest resistance: Plants of the new Chrysanthemum have been observed to be relatively resistant to Fusarium oxysporum f.sp. chrysanthemi and Verticillium dahliae. To date, plants of the new Chrysanthemum have not been observed to be resistant to pests and other pathogens common to plants grown under commercial conditions.
Temperature tolerance: Plants of the new Chrysanthemum have been observed to tolerate temperatures ranging from about −12° C. to 35° C. and to be suitable for USDA Hardiness Zones 8 to 10.

It is claimed:

1. A new and distinct Chrysanthemum plant named ‘DLFSALM2’ as illustrated and described.
